(19) United States
(12) Patent Application Publication
(10) Pub. No.: US 2008/0107699 A1
(43) Pub. Date: May 8, 2008

(54) Method of using topical probiotics for the inhibition of surface contamination by a pathogenic microorganism and composition therefor

(76) Inventors: Mark Spigelman, (US); Mairi Ross, (US)

Correspondence Address: ETHERTON LAW GROUP, LLC, 5555 E. VAN BUREN STREET, SUITE 100, PHOENIX, AZ, 85008, US

(21) Appl. No.: 11/977,371

(22) Filed: Oct. 24, 2007

Related U.S. Application Data
(60) Provisional application No. 60/854,093, filed on Oct. 25, 2006.

(51) Int. Cl. A61K 35/74; A61K 35/00; A61K 36/06; A01N 25/34; A61K 9/70; A61P 17/00

(52) U.S. Cl. 424/404; 424/93.1; 424/93.4; 424/93.45; 424/93.5; 424/93.51; 424/443

(57) ABSTRACT

A method and composition is provided for application of probiotic microorganisms to a surface to prevent contamination by pathogenic microorganisms. The probiotic microorganisms may be bacteria, yeast or mold, such as bacteria of the Lactobacillus genus. Application of probiotic microorganisms may be to surfaces such as of the hands, or to other surfaces such as tables, equipment, clothing and the like. Upon application, the probiotic microorganism may display competitive exclusion of the pathogenic microorganism.

### CROSS-REFERENCE TO RELATED APPLICATIONS

This application claims the benefit of co-pending U.S. Provisional Application No. 60/854,093 filed Oct. 25, 2006.

### FIELD OF THE INVENTION

This invention relates to probiotic microorganisms, particularly for the prevention or inhibition of microbial contamination and cross contamination in health care and other facilities.

### BACKGROUND

Despite efforts made in hygiene and cleanliness, hospitals and other facilities are experiencing contamination and cross contamination problems with pathogenic bacteria, especially methicillin resistant Staphylococcus aureus (MRSA). Such antibiotic resistant bacteria are a persistent problem despite increased attempts to fight cross-contamination and resistant bacteria. A particular problem arises from current methods of preventing contamination such as by frequent scrubbing and hand washing with soap, alcohol or antimicrobial solutions. These preventative actions temporarily eliminate pathogenic microorganisms on skin, but also reduce protective skin commensals, damage skin thereby actually increasing the risk of cross contamination.

Scrubbing is effective in removing pathogens and commensals, but can then leave the skin vulnerable to colonization by pathogenic organisms, particularly the more dangerous forms of antibiotic resistant bacteria that are increasingly creating havoc in our health care institutions, such as MRSA. Scrubbing and washing of hands damages the skin, such as changing skin pH and reducing fatty acids, thereby resulting in changes in normal resident flora that would normally be protective. Numbers of organisms shed from damaged skin are often higher than from normal, healthy skin. It has been shown that the number of organisms spread from the hands of nurses who washed frequently with an antimicrobial soap increases over time. It would be desirable to have a way to clean one's hands, especially those of health care providers, that helps prevent cross-contamination. Bacteria have more difficulty in colonizing any area where there are other bacteria. As in nature, bacteria compete for food and space. Thus where there are more than one bacteria present, it is always more difficult for any single bacterium to become the dominant strain. It follows that antibiotic-resistant bacteria prefer a clean bacteria-free surface to become the main strain on any surface. Constant scrubbing with antiseptics gives them such a surface.

Lactic acid bacteria (LAB), particularly those classified to the Lactobacillus and Lactococcus genera, are often included in foods or dietary supplements and used to treat the small or large intestine for various benefits relating to immune, digestive, urinary and vaginal health. They are considered probiotic which, as used herein, means microorganisms which when administered in adequate amounts are capable of conferring a health benefit on the host. Human skin has its own normal cutaneous microbiota, including propionibacteria. Hypothesizing that the probiotic principle is likely to be applicable to any environment where a normal microbiota exists, Ouwehand et al. conducted a study published in 2003 of the adhesion of dairy strains of propionibacteria to skin and their effect on skin pathogens. Unfortunately, the studied propionibacteria were found to exhibit no inhibition of skin pathogens. Ouwehand et al. (2003) Lett Appl Microbio 36(5):327-331. It would be desirable to have a topical probiotic to maintain a layer of protective propriotic bacteria to prevent the contamination and cross-contamination with pathogenic microorganisms.

Thus, there is a need for improved prevention of contamination and cross contamination of pathogenic microorganisms, especially MRSA and other drug resistant microorganisms. It is an object of this invention to provide a method for the prevention or inhibition of the contamination of a surface by pathogenic organisms by applying one or more probiotic organisms. It is a further object to provide such a method can that can be applied to human hands and hospital surfaces to prevent cross-contamination, especially for drug-resistant bacteria. It is a further object to provide compositions and articles for conveniently and safely implementing the method.

### SUMMARY

The present invention is a method and composition for application of probiotic microorganisms to a surface to inhibit contamination by pathogenic microorganisms. The probiotic microorganisms may be bacteria, yeast or mold. Upon application, the probiotic microorganism may display competitive exclusion of the pathogenic microorganism.

The method involves applying one or more probiotic microorganisms to a surface in an amount effective to at least partly prevent contamination of the surface by the pathogenic microorganism. The preferred probiotic microorganisms are bacteria of the genus Lactobacillus. In a preferred embodiment, the surface is human hands. The hands are washed thoroughly and a lotion containing probiotic microorganisms is applied to the hands. In an alternative embodiment, the surface is that of hospital equipment, and the probiotic microorganisms are applied by spraying the surface of the equipment with an aerosol containing the probiotic organisms. In an alternative embodiment, the surface susceptible to contamination by the pathogenic microorganism is wiped with a paper wipe comprising the probiotic. Eliminating such surface contamination thereby prevents or inhibits cross-contamination to other surfaces.

### DETAILED DESCRIPTION

The present invention provides a method and composition for applying probiotic microorganisms to surfaces such as human skin and hospital equipment and fixtures, to thereby at least partly inhibit the contamination, colonization, growth and cross-contamination of pathogenic microorganisms on hands or other surfaces. This has particular application to biological surfaces that have been stripped or depleted of their protective, commensal bacteria.

Although not wishing to be bound by theory, the inhibitory activity provided by the method and composition may arise from the probiotic microorganisms forming isolated colonies. Pathogenic bacteria generally do not grow on top of other bacteria, so the probiotic microorganisms effectively form a barrier by competitive exclusion that inhibits pathogenic bacteria from growing on the surface. Inhibition therefore, as used herein, includes prevention.

Accordingly, application of sufficient amounts of probiotic microorganisms for a sufficient time may facilitate formation of a layer of protective probiotic microorganisms. Some probiotic microorganisms may also exert antimicrobial activity, such as through release of anti-microbial compounds. The method and composition may be applied to any surface susceptible to contamination by pathogenic microorganisms. Such surfaces include biological surfaces such as skin, or non-biological surfaces such as on tables, benches, charts, hospital fixtures such as door handles and light switches, equipment, utensils, beds, bedding, clothing and the like.

In a preferred embodiment of the method one or more probiotic microorganisms is applied to a person's hands in an amount effective to at least partly inhibit contamination of the surface by one or more pathogenic microorganisms. For example, a surgeon thoroughly scrubs his hands pre-surgery, as known in the art, and then rubs his hands with a composition containing probiotic microorganisms. The surgeon could apply the composition to his hands periodically throughout the day, outside the operating theatre, as he visits his hospital patients. In an alternative embodiment, a nurse periodically dips her hands in a bath containing probiotic microorganisms. In another embodiment, hospital surfaces, such as charts, pens, door handles, telephone handsets, bed frames and the like, are sprayed with an aerosol composition containing probiotic microorganisms. Preferably the surfaces are cleaned prior to spraying, so that the probiotic organisms reside on the surface to the exclusion of pathogenic organisms. In another embodiment, the hospital surfaces are wiped with a paper or fabric wipe that contains probiotic organisms. In this embodiment, the wiping action could serve a dual purpose of cleaning the surface and depositing probiotic microorganisms at the same time.

Probiotic microorganisms may be applied to a surface for a sufficient time to inhibit contamination by pathogenic microorganisms. Sufficient time is dependent upon such factors as the therapeutically effective amount of probiotic microorganisms applied, the type of probiotic microorganisms applied, the mode of application or the degree of contamination of the surface to which the probiotic microorganisms are applied, although without limitation thereto.

A single probiotic microorganism or a plurality of different probiotic microorganisms may be used according to the method or composition. The plurality maybe used serially, in layers, to fight multiple types of pathogenic organisms, or in layers to fight an increasingly resistant single type of pathogenic organism. One probiotic may be subsequently pushed out by a subsequent, more beneficial or less pathogenic probiotic.

In embodiments relating to skin, particularly the skin surface of human hands, the method and composition may reduce frequency of hand washing because contamination is reduced; may reduce damage to skin by reducing the need for excessive hand washing; may maintain normal health of the skin with probiotic bacteria; and may reduce contamination of self and others.

The preferred probiotic microorganisms are bacteria of the genus Lactobacillus. Lactobacillus bacteria are known and readily available to the public from various commercial suppliers. The preferred bacteria can be ordered from Danisco by specifying genus, species and strain number such as: Lactobacillus acidophilus NCFM®; Lactobacillus acidophilus La-14; or Lactobacillus paracasei Lpc-37. Danisco's contact information is on its website at www.danisco.com, and its present mailing address from which product can be ordered is Danisco USA Inc., 3329 Agriculture Drive, Madison, Wis. 53716. Other commercially-available strains are well-known and readily available. Appendix 1 lists a number of probiotic organisms and their commercial suppliers.

Probiotic bacteria may be of any suitable type, including but not limited to the aforementioned Lactobacillus genus including, but not limited to the Lactobacillus acidophilus, Lactobacillus plantarum, Lactobacillus salivarius, Lactobacillus delbrueckiil, Lactobacillus rhamnosus, Lactobacillus bulgaricus, Lactobacillus crispatus, Lactobacillus gasseri, Lactobacillus jensenii; the Lactococcus genus including Lactococcus lactis (subsp. Lactis); Streptococcus thermophilus; Propionibacterium freudenreichii subsp. Shermanii; Enterococccus genus, including Enterococcus faecium and Enterococcus thermophilus; the Bifidobacterium genus, including Bifidobacterium longum, Bifidobacterium infantis, and Bifidobacterium bifidum; Bacillus genus, including Bacillus coagulans, Bacillus thermophilus, Bacillus laterosporus, Bacillus subtilis, Bacillus megaterium, Bacillus licheniformis, Bacillus mycoides, Bacillus pumilus, Bacillus lentus, Bacillus cereus and Bacillus circulans; Sporolactobacillus genus; Micromonospora genus; Micrococcus genus; Rhodococcus genus; Escherichia coli.; and Pseudomonas genus, including Pseudomonas fluorescens and Pseudomonas aeruginosa. The Pseudomonas are soil-based organisms and tend to be hardy, which may be especially advantageous for a topical composition, although some such as Pseudomonas aeruginosa are already pathogenic nosocomials.

Some probiotics are more effective than others, depending on the nature of the pathogen, environment, surfaces, and degree of infection. Similarly some are more benign than others. It is contemplated that certain probiotics that are potentially pathogenic or otherwise malicious, such as spore formers such as B. coagulans, may be desirable because they can survive well in adverse conditions. One probiotic may be subsequently pushed out by a subsequent, more beneficial or less pathogenic probiotic.

As used herein, probiotic microorganisms broadly include bacteria, yeast or mold. Probiotic yeast may be of any suitable type, including but not limited to the genus Saccharomyces, such as described in U.S. Pat. No. 6,524,575. Probiotic mold may be of any suitable type, including but not limited to the genus Aspergillus, such as described in U.S. Pat. No. 6,368,591.

Suitable probiotic microorganisms may be selected according to one or more particular properties. A preferred property is that the probiotic microorganisms display competitive exclusion of pathogenic organisms from the surface to which they are applied. By way of example, other properties may be selected from the group consisting of adherence to human tissue; sensitivity to antibiotics; antimicrobial activity; acid tolerance; and a high oxygen tolerance (such as tested by Relative Bacterial Growth Ratio method).

Non-limiting examples of probiotic microorganisms with high adherence to intestinal cells (in vitro) include Lactobacillus casei Shirota obtained from Yakult Singapore Pty., Ltd., and Lactobacillus rhamnosus GG (ATCC 53103) commercially available from Valio, Helsinki, Finland, Lactobacilllus acidophilus LA-1 commercially available from Chr. Hansen, Milwaukee, Wis. Non-limiting examples of probiotic microorganisms with high adherence to intestinal mucus (in vitro) include Lb. rhamsosus GG, Lc. lactis subsp. lactis and P. freudenreichii subsp. shermanii JS, which are commercially available from Valio, Helsinki, Finland.

Non-limiting examples of probiotic microorganisms that have an anti-microbial affect on pathogenic microorganisms include: Lactobacillus paracasei Lpc-37 which has been shown (in vitro) to inhibit the growth of S. aureus; and Lactobacillus casei Lc-11; which are commercially available from Danisco, USA, Madison, Wis.

Non-limiting examples of acid tolerant probiotic bacteria include: Lactobacillus acidophilus La-14; Lactobacillus casei Lc-11; Lactobacillus paracasei Lpc-37; Lactobacillus plantarum Lp-115; and Lactobacillus rhamnosus L4-32; which are available from Danisco, USA, Madison, Wis.

Non-limiting examples of high oxygen tolerant probiotic bacteria include: B. lactis Bb12, (Chr. Hansen, Denmark) and B. lactis HN019 (DR10) which are commercially available from Danisco, USA, Madison, Wis.

Alternatively, if not available commercially, isolation, identification and culturing of probiotic microorganisms can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980).

Typically, probiotic microorganisms are grown on media that enhance viability of the microorganisms. Examples include, but are not limited to, protein milk digest and MRS-cysteine.

Generally, probiotic microorganisms may be viable or non-viable or may be in the form of a spore (“sporolated”) or lyophilized. Maintenance of viability of probiotic cells may be facilitated by encapsulation. A particular example is encapsulation of probiotic bacterial cells within a sesame oil emulsion. A non-limiting example of sesame oil encapsulation is described by Hou, 2003, J Dairy Sci, 86:424-428, where sesame oil encapsulation elevated viability from 0.023 to 5.45%. Sesame oil encapsulated bacteria can demonstrate a significant increase in viability (approximately 104 times) when subjected to a high acid environment. In other non-limiting examples, encapsulation may be by way of oxygen impermeable containers, microencapsulation, alginate encapsulation or polysaccharide matrix encapsulation. Viability may also be facilitated by incorporation of nutrients such as peptides and amino acids. Non-viable probiotic microorganisms may be “killed,” e.g thermally killed cells, cells killed by exposure to altered pH, chemicals such as phenol or elevated pressure. Non-viable probiotic microorganisms may be simpler to produce and store. Methods of generating spores and lyophilization are well known in the art.

Probiotic microorganisms may be applied in the form of a composition. The carrier may be of any suitable type that does not substantially interfere with, inhibit or negate the pharmacological activity or the viability of probiotic microorganisms in the composition. For example, a composition may be in the form of a liquid, an emulsion, a cream, a lotion, a paste, a gel, an oil, an ointment, a suspension, an aerosol spray, a powder, or a semi-solid. Suitable carriers will be compatible with the surface to which the composition is to be applied.

A preferred composition takes the form of an oil-based lotion. An oil base preserves viability of cells better than a water base, although a water-based composition may appropriate in certain situations. A version is a sesame oil-based lotion prepared according to the following recipe. Sesame oil bodies are extremely stable because of the steric hindrance and electronegative repulsion provided by oleosins on their surfaces. The compressed oil bodies of mature seed never coalesce or aggregate. This makes them an excellent matrix oil for probiotic bacteria. A study by Hou showed that probiotic bacteria in artificial sesame oil emulsion had superior viability. The lotion is prepared by obtaining mature sesame seeds and extracting oil bodies according to the procedure reported by Tzen at al (1997). Sesame varieties and seeds are commercially available from Sesaco Corporation. San Antonio, Tex. 78217. Heat sesame oil bodies to 70 degrees C. for one hour to decompose. Cool decomposed oil bodies (approx 50 μl) to 40 degrees C. and then mix with 10, 25, 50, or 100 times the amount of decomposed bodies of commercial oil (vegetable or mineral) but preferably 50 times. Vortex the mixture for 15 minutes to mix the bacteria into the reconstituted oil emulsions and add Lactobacillus acidophilus NCFM® bacteria at 2.2×10⁸ CFU per ml.

Another version comprises 10⁹ CFU Lactobacillus acidophilus NCFM® bacteria and 1 ml olive oil, grape seed oil, sesame, sweet almond oil or other oil, vortexed for 15 minutes at ambient temperature.

Another version enables probiotic organisms to be added to a standard cosmetic formula set forth by Flick E W, Cosmetic and Toiletry Formulations 2^{nd} Edition Volume 3 as its Moisturizing Lotion (Cold Preparation). The following ingredients are used, pursuant to the recipe below:

| Ingredients | |
|---|---|
| Group A | % by weight |
| Water | 90.00 |
| Hydroxypropylmethylcellulose (Methocel 40-100) | 0.10 |
| AMP | 0.40 |
| Oxaban-A | 0.10 |
| Glycerin | 2.50 |
| Disodium EDTA | 0.10 |

| Group B | % by weight |
|---|---|
| White Mineral Oil (Drakeol 9) | 3.00 |
| White Petrolatum (Penreco Snow) | 3.00 |
| Dimethicone (Silicone SF 96-200) | 0.50 |
| Isopropyl Palmitate | 1.00 |
| Sodium Isethionate (Hostapon KA) | 0.10 |
| Pemulen TR-2 | 0.20 |

Production is conducted at ambient temperature. All ingredients are sterile until the addition of the probiotic bacteria.
1. Combine Group A ingredients except Hydroxypropylmethylcellulose.
2. Vigorously agitate and add Hydroxypropylmethylcellulose
3. In a separate vessel, combine Group B ingredients. Mix until uniform.
4. Add 3 to 2 with vigorous agitation.
5. Add probiotic bacteria Lactobacillus acidophilus NCFM® contained in liposomes and vigorously agitate 15 minutes.

In another embodiment, the composition is a spray containing the probiotic organisms. 10 mg Lactobacillus acidophilus NCFM® at a concentration of 100 billion CFU per gram and 40 mg water soluble fructooligosaccharide are blended together and added to 1 cc of distilled water in a spray bottle.

In another embodiment, the surface of the equipment is wiped with a paper wipe comprising the probiotic organisms. 200 mg Lactobacillus acidophilus NCFM® at a concentration of 100 billion CFU per gram is mixed with 800 mg water-soluble fructooligosaccharide. The probiotic mixture is sprinkled on a sheet of flexible, water-permeable material, such as filter paper or gauze, and a second sheet of similar material is fastened to the first sheet to encase the probiotic organism mixture. When ready to use, the wipe is dampened and rubbed on the desired surface.

In certain embodiments where the composition is for application to the skin of a human host, the carrier should be acceptable to humans. Terms such as “physiologically acceptable carrier” and “pharmaceutically acceptable carrier” may be interchangeably used and refer to a carrier or a diluent that does not cause significant irritation to a host and does not abrogate the biological activity and properties of the administered compound. An adjuvant is included under these phrases.

Carriers may be solid-based, dry materials. Typical carriers for dry formulations include, but are not limited to, trehalose, malto-dextrin, rice flour, micro-crystalline cellulose (MCC), magnesium stearate, inositol, fructo-oligosaccharides (FOS), gluco-oligosaccharide (GOS), dextrose, sucrose, and the like. Where the composition is dry and includes evaporated oils that may cause the composition to cake (i.e, adherence of the component spores, salts, powders and oils), it is preferred to include dry fillers, which distribute the components and prevent caking and enhance its application to the skin. Exemplary anti-caking agents include MCC, talc, diatomaceous earth, amorphous silica, gelatin, saccharose, skimmed dry milk powder, starch and the like, which are typically added in an amount of from approximately 1% to 95% by weight. It will be appreciated that dry formulations, which are subsequently rehydrated are preferred to initially hydrated formulations.

Alternatively, the carrier may be suitable for formulation into a liquid or gel form. Suitable liquid or gel-based carriers include but are not limited to, water and physiological salt solutions; urea; alcohols and derivatives (e.g., methanol, ethanol, propanol, butanol); glycols (e.g, ethylene glycol, propylene glycol, and the like). Preferably, carriers have a neutral pH (i.e., about pH 7.0), particularly when in liquid or gel form.

In other embodiments, carriers are aqueous and oleaginous carriers. Such carriers include, for example, mineral oil, sesame oil, almond oil, lanolin alcohols, sorbitan mono-oleate, fragrant or essential oils, or mixed with water to form a lotion, gel, cream or semi-solid composition.

The composition may further comprise an excipient. As used herein, the term “excipient” refers to an inert substance added to a composition to further facilitate administration of the composition. Non-limiting examples include various sugars and types of starch, cellulose derivatives, gelatin, vegetable oils and polyethylene glycols.

The composition may further comprise one or more preservatives. Non-limiting examples of preservatives include methylparaben, propylparaben, benzyl alcohol and ethylene diamine tetraacetate salts.

The composition may include a plasticizer such as glycerol or polyethylene glycol (PEG). Polyethylene glycol (PEG) is a biocompatible polymer with a wide range of solubility in both organic and aqueous media. A preferred molecular weight of PEG is MW=800 to 20,000.

In embodiments where liquid-based compositions containing spores are provided, it is preferable to include a spore germination inhibitor to promote long term storage. Any spore germination inhibitor may be used. Preferred inhibitors include: hyper-saline carriers, methylparaben, guar gum, polysorbates, preservatives, and the like

Compositions may further include one or more agents such as a nutrient, an anti-fungal agent, an antibiotic, an antioxidant (e.g. vitamin E), a plant extract (e.g. Aloe Vera), a buffering agent, an oil, a lubricant (e.g. synthetic or natural beeswax, lanolin), a moisturizer, a coloring agent, a flavoring, a vitamin or a mineral, which may be selected according to the intended use or the route of administration employed.

Compositions may be formulated according to the intended use of the composition, as is well understood by persons skilled in the art. With regard to the method and composition, toxicity, safety and therapeutic efficacy can readily be determined by a person of ordinary skill in the art using standard testing procedures in vitro, in cell cultures or in experimental animals. Data obtained can be used in formulating suitable dosages and application regimes as required.

By way of example only, a review of formulation techniques can be found in “The Theory and Practice of Industrial Pharmacy” (Ed. Lachman L. et al., 1986) and Laulund in “Commercial aspects of formulation, production and marketing of probiotic products”. (Gibson, S. (Ed.) 1994). Reference is also made to “Remington's Pharmaceutical Sciences,” Mack Publishing Co., Easton, Pa., which provides non-limiting examples of techniques for formulation and administration of compositions.

In an embodiment, the composition may be provided in a dispenser to facilitate delivery of the composition. The dispenser may be a pump, aerosol spray, squeezable tube, soap dispenser, tub, blister pack or the like. Dosing and application can be in the form of a single or multiple doses.

Suitably, a therapeutically effective amount of probiotic microorganisms is used. A “therapeutically effective amount” of one or more probiotic microorganisms means an amount effective to inhibit pathogenic microbial contamination of the surface to which the one or more probiotic microorganisms are to be applied. Therefore, the upper limit is typically bounded by cost or manufacturing considerations, but not necessarily the amount necessary to inhibit contamination. “Amount” may be expressed in terms of percent (%) by weight, viable cell count, antimicrobial activity (for example Minimal Inhibitory Concentration) or any other measure known in the art. By way of example only, probiotic microorganisms may constitute 1-90%, more preferably 5-90%, even more preferably 10-90% or still more preferably 15-88% by weight of the composition. By way of example only, at least 106 viable probiotic microorganisms are provided per dose and preferably at least 1010 viable probiotic microorganisms are provided per dose at time of manufacture.

An article of manufacture may comprise a composition comprising one or more probiotic microorganisms and packaging material. A carrier or dispenser may also be provided. For example, compositions of the present invention may, be presented in a pack, such as an FDA approved kit, which may contain one or more unit dosage forms containing the probiotic microorganisms. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration of an approved product insert.

The composition may be included in a product identified for the application such as described above. Typically, the product is in the form of a package containing the probiotic bacterial cells or compositions including same, or in combination with packaging material. The packaging material is selected to retain bacterial viability and includes a label or instructions for, for example, use of the components of the package. The instructions indicate the contemplated use of the packaged component, as described herein for the methods or compositions of the invention, contents (e.g., genus, species, strain designation), minimum numbers of viable bacteria at end of shelf-life, proper storage conditions and corporate contact details for consumer information. The label may also provide information related to the freshness of the product. This information may include a date of manufacture, a “sell by” date or a “best before date”. A “sell by” date specifies by which date the product should have been sold to the consumer. A “best before” date specifies by when the product should be disposed of by vendor or consumer.

Alternatively or additionally active labeling may be used. For example, U.S. Pat. Nos. 4,292,916, 5,053,339 5,446,705 and 5,633,835 describe color changing devices for monitoring the shelf-life of perishable products. These devices are initiated by physically bringing into contact reactive layers so that the reaction will start, and this action can only conveniently be performed at the time of packaging. This approach is suitable for monitoring the degradation of foodstuffs which lose freshness throughout the entire distribution chain. U.S. Pat. No. 5,555,223 describes a process for attaching timing indicators to packaging, including the step of setting the timer clock at the exact time of production.

In another aspect, a method of inhibiting contamination by a pathogenic microorganism includes applying one or more probiotic microorganisms to an article and contacting the article with a surface susceptible to contamination by the pathogenic microorganism to at least partly inhibit contamination by the pathogenic microorganism.

For example, one or more probiotic microorganisms, or a composition comprising same, may be applied to an article that is intended to be worn or attached to skin of a human, or other surface, to allow probiotic activity of the microorganisms to occur adjacent to, or on the skin or other surface, in order to inhibit contamination or cross-contamination by pathogenic microorganisms.

The article may be a flexible or pliable material. Examples of flexible or pliable material include a skin wipe, dermal patch, adhesive tape, absorbent pad, or article of clothing.

In another embodiment, one or more probiotic microorganisms, or a composition comprising same, may be applied to an article comprising a solid matrix by impregnation into the solid matrix. The solid matrix may be a fibrous or non-fibrous matrix.

The invention described herein is particularly, although not exclusively, suitable for prevention or inhibition of contamination and cross-contamination by pathogenic microorganisms in the context of human health care facilities (such as hospitals and surgeries) and human health care professionals. It will also be understood that the invention is applicable to other environments where contamination and cross-contamination by pathogenic microorganisms can be a problem. These include infant welfare centers, veterinary health centers, gym and sports locker rooms, restaurants and other food preparation areas, schools, old age and nursing homes, and private homes.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

APPENDIX 1
Commercial Probiotic Strains
This table lists some commercial probiotic strains currently available. Probiotic species
are listed as reported by manufacturer. This speciation may not reflect the most current
taxonomy.

| Strain | Commercial products | Source |
|---|---|---|
| L. acidophilus NCFM ® | Sold as ingredient | Danisco (Madison WI) |
| B. lactis HN019 (DR10) | | |
| L. rhamnosus HN001 (DR20) | | |
| Saccharomyces cerevisiae | Florastor | Biocodex (Creswell OR) |
| (boulardii) | | |
| B. infantis 35264 | Align ® | Procter & Gamble (Mason OH) |
| L. fermentum VRI003 (PCC) | Sold as ingredient | Probiomics (Eveleigh, |
| | | Australia) |
| L. rhamnosus R0011 | Sold as ingredient | Institut Rosell (Montreal, |
| L. acidophilus R0052 | | Canada) |
| L. acidophilus LA-1 | Sold as ingredient | Chr. Hansen (Milwaukee |
| L. paracasei CRL 431 | | WI) |
| B. lactis Bb-12 | Good Start Natural | Nestle (Glendale, CA) |
| | Cultures ® infant | |
| | formula | |
| L. casei Shirota | Yakult ® | Yakult (Tokyo, Japan) |
| B. breve strain Yakult | | |
| L. casei DN-114 001 (“L. casei | DanActive ® | Danone (Paris, France) |
| Defensis ™” | fermented milk | |
| B. animalis DN173 010 | Activia ® yogurt | Dannon (Tarrytown, NY) |
| (“Bifidis regularis ™”) | | |
| L. reuteri RC-14 ™ | Femdophilus ® | Chr. Hansens (Milwaukee |
| L. rhamnosus GR-1 ™ | | WI) |
| | | Urex Biotech (London, |
| | | Ontario, Canada) |
| | | Jarrow Formulas (Los |
| | | Angeles, CA) |
| L. johnsonii Lj-1 (same | LC1 ® | Nestlé (Lausanne, |
| as NCC533 and formerly | | Switzerland) |
| L. acidophilus La-1) | | |
| L. plantarum 299V | Sold as ingredient | Probi AB (Lund, Sweden) |
| L. rhamnosus 271 | | |
| L. reuteri ATCC 55730 | Stonyfield Farms | Biogaia (Stockholm, |
| (“Protectis”) | yogurts | Sweden) |
| L. rhamnosus GG (“LGG”) | Culturelle ®; Dannon | Valio Dairy (Helsinki, |
| | Danimals ® | Finland) |
| L. rhamnosus LB21 | Sold as ingredient | Essum AB (Umeå, |
| Lactococcus lactis L1A | | Sweden) |
| L. salivarius UCC118 | | University College (Cork, |
| | | Ireland) |
| B. longum BB536 | Sold as ingredient | Morinaga Milk Industry |
| | | Co., Ltd. (Zama-City, |
| | | Japan) |
| L. acidophilus LB | Sold as ingredient | Lacteol Laboratory |
| | | (Houdan, France) |
| L. paracasei F19 | Sold as ingredient | Medipharm (Des Moines, |
| | | Iowa) |
| L. paracasei LP-33 | Sold as Ingredient | GenMont Biotech |
| | | (Taiwan) |
| Lactobacillus acidophilus La5* | | Chr. Hansen, Hørsholm, |
| | | Denmark |
| Lactobacillus casei Shirota† | | Yakult, Tokyo, Japan |
| Lactobacillus johnsonii V LA1† | | Nestlé, Lausanne, |
| | | Switzerland |
| Lactobacillus paracasei-33‡ | | Uni-President Enterprises |
| | | Corp., Tainan Hsien, |
| | | Taiwan |
| Lactobacillus plantarum† | | American Type Culture |
| | | Collection (ATCC 8014) |
| Lactobacillus reuteri ING1‡ | | Ingmanfoods, Söderkulla, |
| | | Finland |
| Lactobacillus rhamnosus GG† | | Valio, Helsinki, Finland |
| Lactococcus lactis subsp. lactis† | | Valio, Helsinki, Finland |
| Enterococcus faecium† | | Arla Foods, Viby, |
| | | Denmark |
| E. faecium SF68‡ | | Oriola, Espoo, Finland |
| Propionibacterium | | Valio, Helsinki, Finland |
| freudenreichii subsp. shermanii | | |
| JS† | | |
| Lactobacillus acidophilus La5* | | Chr. Hansen, Hørsholm, |
| | | Denmark |
| Lactobacillus casei Shirota† | | Yakult, Tokyo, Japan |

We claim:

1. A method for the inhibition of the contamination of a surface by a pathogenic microorganism, the method comprising:
a) applying one or more probiotic microorganisms to the surface in an amount effective to at least partly inhibit contamination of the surface by the pathogenic microorganism.

2. The method of claim 1 wherein the probiotic microorganism displays competitive exclusion of the pathogenic microorganism.

3. The method of claim 2 wherein the probiotic microorganism further displays one or more properties selected from the group consisting of: adherence to human tissue; acid tolerance; sensitivity to antibiotics; antimicrobial activity; and high oxygen tolerance.

4. The method of claim 1 wherein the surface is human skin.

5. The method of claim 3 wherein the probiotic microorganism is applied to the human skin after washing.

6. The method of claim 3 wherein the probiotic microorganism is applied to the human skin periodically.

7. The method of claim 1 wherein the probiotic microorganism is a bacterium.

8. The method of claim 7 wherein the bacterium is of the Lactobacillus genus.

9. The method of claim 8 wherein the bacterium is selected from the group consisting of: Lactobacillus acidophilus, Lactobacillus plantarum, Lactobacillus salivarius, Lactobacillus delbrukil, Lactobacillus rhamnosus, Lactobacillus bulgaricus, Lactobacillus gasseri and Lactobacillus jensenii.

10. The method of claim 1 wherein the probiotic microorganism is a yeast or mold.

11. A method of inhibiting contamination by a pathogenic microorganism comprising:
a) applying one or more probiotic microorganisms to an article; and
b) contacting the article with a surface susceptible to contamination by the pathogenic microorganism to at least partly inhibit contamination by the pathogenic microorganism.

12. The method of claim 11, wherein the surface is human skin.

13. The method of claim 11, wherein the article is a skin wipe, a dermal patch, an adhesive tape, an absorbent pad, or an article of clothing.

14. A composition applicable to a surface susceptible to contamination by a pathogenic microorganism, the composition comprising:
a) one or more probiotic microorganisms in an amount effective to at least partly inhibit contamination of the surface by the pathogenic microorganism.

15. The composition of claim 14 which is topically applicable to human skin.

16. The composition of claim 14 further comprising an agent selected from the group consisting of: a preservative; an antioxidant; a nutrient; a buffering agent; a lubricant; a preservative; a moisturizer, an oil; a fragrance; an antibiotic; an anti-fungal agent, a plant extract, a coloring agent, a flavoring, a vitamin and a mineral.

17. The composition of claim 14 wherein the probiotic microorganism constitutes 10-90% by weight of the composition.

18. The composition of claim 14 wherein the probiotic microorganism displays one or more properties selected from the group consisting of: adherence to human tissue; acid tolerance; sensitivity to antibiotics; antimicrobial activity; competitive exclusion of pathogenic organisms; and high oxygen tolerance.

19. The composition of claim 14 wherein the probiotic microorganism is a bacterium.

20. The composition of claim 19, wherein the bacterium is of the Lactobacillus genus.

21. The composition of claim 19, wherein the bacterium is selected from the group consisting of: Lactobacillus acidophilus, Lactobacillus plantarum, Lactobacillus salivarius, Lactobacillus delbrueckii, Lactobacillus rhamnosus, Lactobacillus bulgaricus, Lactobacillus gasseri and Lactobacillus jensenii.

22. The composition of claim 19 wherein the bacterium is viable at the time of manufacture.

23. The composition of claim 19 which comprises at least 10⁶ viable bacteria per dose at the time of manufacture.

24. The composition of claim 19 wherein the viable bacteria are encapsulated.
